(19) United States
(12) Patent Application Publication
(10) Pub. No.: US 2013/0156832 A1
(43) Pub. Date: Jun. 20, 2013

(54) EUDERMIC COMPOSITIONS

(71) Applicants: Barbara Onida, Torino (IT); Renato Silvio Mortera, Collegno (TO) (IT)

(72) Inventors: Barbara Onida, Torino (IT); Renato Silvio Mortera, Collegno (TO) (IT)

(73) Assignee: Miso S.r.I., Torino (IT)

(21) Appl. No.: 13/702,250

(22) PCT Filed: Jul. 12, 2011
(86) PCT No.: PCT/IB11/53112

§ 371 Date: Feb. 14, 2013

(30) Foreign Application Priority Data
Jul. 14, 2010 (IT) TO2010A000612

(51) Int. Cl. A61K 9/00

(52) U.S. Cl. CPC A61K 9/0014; Y10S 977/814; Y10S 977/906; B82Y 5/00
USPC 424/401; 514/159; 424/489; 977/814; 977/906

(57) ABSTRACT

Eudermic composition comprising at least one eudermically active principle, at least one vehicle for said active principle, and porous silica particles, wherein the active principle is contained in at least one pore of at least a first portion of such silica particles and in the vehicle.

### FIELD OF THE INVENTION

The present description concerns eudermic compositions destined for dermatologic and cosmetic applications.

### TECHNICAL BACKGROUND

The dermatologic creams currently available are destined both for the care of an ever expanding range of skin diseases and for cosmetic applications.

A majority of the traditional dermatologic creams have active principles that are transported into the body by means of specific components that serve as vehicles for them.

Such creams are administered in a “pulsed” manner, i.e., repeated treatments of the zone of interest are required during the day or over the entire treatment period.

This type of administration is required because the post-application efficacy of traditional creams is limited to a period comprised between 3 and 6 hours.

Repeated treatments result in considerable inconvenience to the patient and often inopportune forgetfulness.

Moreover, many dermatologic pathologies, grouped under the generic name of chronic dermatitis, have a cyclic and recurring character, creating complex treatment problems over the course of the life of the patient.

From the graph in FIG. 1 we see that pulsed administration in addition to providing limited therapeutic action in a short period of time post-application, also has a period of action in which the concentration is inefficacious and a period of overdose that exerts a toxic effect.

The so-called transdermal or patch systems have been developed to increase the efficacy of traditional creams, apparently analogous to the common medicated patches, however capable of storing and releasing a drug over a more or less programmable time interval.

Transdermal systems are multilaminated systems that are applied on the skin by simple pressure, as normal patches.

Various types are commercially available: from the simplest, so-called “flat” patches that release the drug for a brief time period, to more complex forms, composed of four layers.

Such layers are generally constituted of:
an external isolating material,
a reservoir containing the drug,
a porous polypropylene membrane that provides the slow continuous rhythm of release,
an oily solvent that allows the drug to reach the lower layer of the skin where the blood vessels are found.

Such devices offer the great advantage of not being “invasive” for the patients, because they are applied to the skin as a simple patch.

Using these devices, for some active principles it is possible to obtain zero order release at constant skin concentration for an extended time interval (FIG. 1), thus minimising the negative effects of pulsed administration.

Still such patches have the principle inconvenience of having a maximum efficacy of 24 consecutive hours, which increases the duration of traditional creams, but still requires frequent replacement.

A further possibility for overcoming the inconveniences of pulsed administration of traditional creams is provided by the use of specific materials that have physical characteristics that allow for controlled release of the active principle delivered.

Mesoporous materials and in particular mesoporous silica were proposed as vehicles for the controlled release of drug in 2001 by Vallet-Regí et al. (Chem. Mater., 2001; 13:308).

Such materials have pores characterised by a high specific surface area, greater than 1000 m²/g (C. T. Kresge et al., Nature, 1992; 359:710) and by a diameter that can be modulated by varying the synthesis conditions.

Thanks to such characteristics mesoporous materials and in particular mesoporous silica, for example in particulate form, are used to incorporate, deliver and release active principles in various layers.

For example, the international patent application WO-A-2005/009602 describes mesoporous silica particles made to deliver substances such as for example drugs, polynucleotides, polypeptides, hormones, enzymes that are loaded inside the pores of the particle. Also, such particles are characterised in having plugs that function to close the pores to slow or prevent release of the encapsulated agent.

The Japanese patent application JP-A-2009013142 describes mesoporous silica made to contain and deliver active principles across cellular membranes.

The international patent application WO-A-2009/110939 Describes devices useful for the transport and release of bioactive agents, such as for example drugs, in combination with radioisotopes for the chemo- or radio-therapeutic treatment of tumours. Such devices can be of mesoporous silica particles.

Devices comprising mesoporous silica offer the possibility of releasing the drug, but are not resolutive in terms of programming or prolonging their release.

### SUMMARY OF THE INVENTION

Keeping these preambles in mind, the need is thus felt for improved, more efficacious solutions, that allow a eudermic composition to be made that can release at least one active principle in a sustained and prolonged manner.

According to the invention, the above-said object is obtained by means of the solution specifically recalled in the attached claims, which constitute an integral part of the present description.

The present description concerns a eudermic composition comprising at least one active principle localised both inside the porous silica particles and dispersed inside the composition, where the composition is preferably destined for topical application.

In one embodiment, the eudermic composition comprises at least one eudermically active principle, at least one vehicle for the eudermically active principle and porous silica particles, where the eudermically active principle is contained in at least one pore of a portion of such silica particles and in said vehicle.

A second embodiment of the present description concerns a eudermic composition that comprises a eudermically active principle, a vehicle for the eudermically active principle and porous silica particles, where the eudermically active principle is contained in at least one pore of at least a portion of said silica particles and in said vehicle, the composition further comprising at least one further eudermically active principle, at least one further vehicle for such further eudermically active principle, the further eudermically active principle being contained in at least one pore of a second portion of the silica particles and in at least one further vehicle, and the further eudermically active principle being soluble in such further vehicle.

The results reported below show that the previously described eudermic composition has the advantage of maintaining prolonged eudermic action at the site of application, substantially reducing the inconveniences to subjects that use such composition.

### BRIEF DESCRIPTION OF THE FIGURES

The invention will now be described in detail, purely by way of non-limiting example with reference to the annexed drawings, in which:

FIG. 1: Trend over time of the skin concentration of a drug in the case of controlled and non-controlled administration.

FIG. 2: Trend over time of the concentration of salicylic acid (SA). Triangles: control solution. Squares: solution with 5 mg of mesoporous spheres loaded with SA. Circles: solution with 10 mg of mesoporous spheres loaded with SA.

### DETAILED DESCRIPTION OF SOME EMBODIMENTS

The invention will now be described in detail, by way of non-limiting example only, with reference to the realisation of a eudermic composition for the sustained release of the active principle.

In the description that follows, numerous specific details are presented to provide a thorough understanding of the embodiment. The embodiments can be practiced without one or more of the specific details, or with other methods, components, materials, etc. In other instances, well-known structures, materials, or operation are not shown or described in detail to avoid obscuring aspects of the embodiments.

Reference throughout this specification to “one embodiment” or “an embodiment” means that a particular feature, structure or characteristic described in connection with the embodiment is included in at least one embodiment. Thus, the appearances of the phrases “in one embodiment” or “in a certain embodiment” in various places throughout this specification are not necessarily all referring to the same embodiment. Furthermore, the particular features, structures or characteristics may be combined in any suitable manner in one or more embodiments.

The headings provided herein are for convenience only and do not interpret the scope or meaning of the embodiments.

One embodiment of the present description concerns a eudermic composition comprising:
at least one eudermically active principle,
at least one vehicle for said active principle, and
porous silica particles,
   in which said active principle is contained in at least one pore of at least a first portion of said silica particles and in said vehicle.

The active principle is present in the vehicle at a concentration equal to its saturation concentration in such vehicle. In the case in which the eudermic composition contains a second vehicle, such second vehicle is chosen so to not solubilise the active principle contained in the silica particles.

A second embodiment of the present description concerns a eudermic composition that comprises a eudermically active principle, a vehicle for the eudermically active principle and porous silica particles, where the eudermically active principle is contained in at least one pore of at least a portion of such silica particles and in said vehicle, the composition comprising, in addition, at least one further eudermically active principle, at least one further vehicle for such further eudermically active principle, the further eudermically active principle being contained in at least one pore of a second portion of the silica particles and in at least one further vehicle, and the further eudermically active principle being soluble in such further vehicle.

The further eudermically active principle is contained in the further vehicle for such further eudermically active principle at a concentration equal to its saturation concentration in such further vehicle.

The further active principle is insoluble or scarcely soluble in the vehicle in which the first eudermically active principle is dissolved and—in parallel—the first eudermically active principle is insoluble or scarcely soluble in the further vehicle in which the further eudermically active principle is dissolved.

The vehicle/s used in the composition object of the present description can be hydrophilic vehicles or lipophilic vehicles and are chosen in function of the solubility of the active principle/s used in the corresponding vehicle.

Among the hydrophilic vehicles advantageously usable in the present compositions it is possible to cite, by way of non-limiting example, water and alcohols.

Among the lipophilic vehicles advantageously usable in the present compositions it is possible to cite, by way of non-limiting example, paraffin oil, castor oil, vaseline oil.

The compositions object of the present description are capable of maintaining a sustained therapeutic action at the site of application for several days permitting greater treatment efficacy than the products currently on the market, substantially reducing the inconveniences to the patients due to repeated administration of traditional creams.

The compositions object of the present description can be destined for the treatment of pathologies (for example, warts, psoriasis, eczema, mycoses, keratoses) and skin imperfections (for example pigmented lesions) optimising treatment times and avoiding numerous and repeated applications typical of the current care.

Moreover, the silica particles act as protection from the active principles themselves by limiting direct and harmful contact between the drugs loaded and the skin, and avoiding local skin rashes and irritations.

The results obtained by the present Inventors show that the concentration of active principle/s released is maintained constant over an interval of time that can be modulated as needed.

By increasing or decreasing the amount of silica particles present in the composition it is possible to obtain a constant release of the active principle/s, for example at the site of application of the composition, for a period of time comprised between 1 and 10 days.

It was also shown that the silica exerts important functions on the body, such as plastic, trophic, soothing and antioxidant functions.

Silica is present primarily in connective tissues and in particular in the dermis, in the corneal layer of the epidermis, in the hair and nails.

However, it is difficult to assimilate in the naturally occurring form.

The eudermic compositions object of the present description are characterised by a synergistic action of silica and the active principle released in a controlled and continuous manner that leads to greater efficacy with respect to the currently available compositions.

By means of the technology set up for the release of drug by mesoporous silica it is possible to incorporate a quantity of active principle (AP) inside of porous silica particles, preferably silica particles having sub-micrometric sizes, and still more preferably particles with sub-micrometric sizes and controlled porosity.

Such particles can be prepared following different procedures described in the literature (M. Grün et al., Microporous Mesoporous Mat. 27 (1999) 207; C. Y. Lai et al., J. Am. Chem. Soc. 125 (2003) 4451; Y. Yamada and K. Yano, Microporous Mesoporous Mat. 93 (2006) 190) that envision the use of a surfactant as a template (normally hexadecyltrimethylammonium bromide (C₁₆TMABr)) dissolved in a basic solution of water-ethanol-ammonia and a source of silica (normally tetraethyl orthosilicate ((TEOS)).

They have cylindrical pores with hexagonal symmetry and a monodisperse size distribution variable from 2 nm to 50 nm in diameter, according to the synthesis conditions.

Using controlled porosity silica particles with sub-micrometric sizes it is possible to vary the quantity of active principle loaded comprised between 10% and 50% in weight of the particles, according to the porosity chosen and the AP itself.

The loading of active principle in the pores of the mesoporous silica is carried out by placing such particles in contact with a solution containing the AP dissolved at a known concentration.

The solvents used most for loading the AP into the mesoporous silica are ethanol, methanol, pentanol, hexanol, acetone, water and buffer solutions.

The compositions object of the present description can be used either directly on the site of interest with a convenient occlusive dressing or combined with transdermal patches to prolong efficacy beyond 24 hours.

The compositions object of the present description can be administered in the form of cream, pomade, ointment, paste, gel or be contained in a transdermal release system.

Below indications will be provided for the realisation of a eudermic composition comprising a single eudermically active principle. However, it is evident that such indications are given purely by way of example, it being possible to realise—according to the indications provided herein—compositions containing more than one active principle.

In a particular embodiment, the particles containing the AP are dispersed in a eudermic composition comprising two vehicles:

a first vehicle, in which the active principle is soluble (named vehicle A, for example water), and

a second vehicle, in which the active principle is not soluble or is scarcely soluble (named vehicle B, for example paraffin oil).

In this way it is possible to control the concentration of active principle present in the eudermic composition by acting exclusively on the quantity of active principle dissolved in vehicle A (M_{AP}).

In particular, if the concentration of AP in the vehicle A (C_{AP,v.A}) is maintained equal to the saturation concentration or solubility (C_{S,AP-A}), it is possible to modify the concentration in the eudermic composition (C_{AP,c.e.}) by simply varying the volume of vehicle A (V_{A}) with respect to vehicle B (V_{B}), as described in the following equations:

C_{PA, v.A}=M_{PA}/V_{A}=C_{S,PA-A} (1)

C_{AP,c.e}=M_{AP}/(V_{A}+V_{B}) (2)

from which:

C_{AP, c.e.}=V_{A}*C_{S,AP-A}/(V_{A}+V_{B}) (3)

Dispersing the silica particles containing the AP in the eudermic composition prepared at the C_{AP,c.e.} desired, they act as a reservoir of AP, maintaining the concentration of AP in the vehicle A (C_{AP,v.A}) equal to its saturation concentration (solubility) until the pores are completely empty.

In this way it is possible to maintain the AP concentration in the eudermic composition constant for a time that can be modulated according to necessity, by increasing or decreasing the amount of silica particles containing the AP.

### Example 1

### Salicylic Acid (SA)-Based Dermatologic Cream for the Treatment of Warts at a Concentration of 0.11 mg/ml

In 100 ml of cream constituted of a vehicle A (water) and a vehicle B (paraffin oil in which SA is scarcely soluble), the total volume of which is given by the sum of the volume of vehicle A (V_{A}) and the volume of vehicle B (V_{B}), the amount of salicylic acid is 11 mg (M_{SA}).

According to equation (3):

0.11 mg/ml=*2 mg/ml/100 ml

from which:

V_{A}=5.5 ml and V_{B}=94.5 ml

To the 100 ml of cream are added 150 mg of sub-micrometric silica particles having 2 nm pores ordered in a hexagonal symmetry, prepared using hexadecyltrimethylammonium bromide (C₁₆TMABr) as a template and tetraethyl orthosilicate (TEOS) as a silica source (M. Grün et al., Microporous Mesoporous Mat. 27 (1999) 207).

Such particles are loaded with an amount of SA equal to 20% of their weight (30 mg), placing them in contact with a 0.5 M solution of SA in methanol for 4 hours at room temperature with continuous agitation.

In this way it is possible to maintain the concentration of SA constant for a period about 3 times longer than the creams currently marketed.

In addition, it is possible to increase the quantity of silica particles to further prolong the effect of SA over time, up to 30 times longer with respect to the creams without silica particles, i.e., up to about 10 days.

### Example 2

### Control of Salicylic Acid (SA) Concentration in Vehicle A (Water)

Three separate solutions of salicylic acid in 3 ml of water (vehicle A) were prepared at the saturation concentration (C_{s, SA-A}=2 mg/ml).

The first solution served as a control containing only SA (6 mg).

To the second solution 5 mg of particles were added, containing 20% SA by weight (1 mg).

To the third solution 10 mg of particles were added, containing 20% SA by weight (2 mg).

Every hour for the first three hours, 0.5 ml of solution was removed and replaced by 0.5 ml of double distilled water to simulate absorption of SA by the skin.

After 4 and 5 hours 2 ml of solution were removed and replaced by 2 ml of double distilled water.

After each removal, the concentration of SA in the solution was evaluated by means of a UV-Vis spectrophotometer (Cary 500 Scan, λ=297 nm).

FIG. 2 reports the concentration trend of SA over time for the three solutions.

It can be seen that the two solutions containing the mesoporous silica particles maintain a constant SA concentration equal to the saturation concentration (C_{S, SA-A}=2 mg/ml) until the pores containing the reservoir of active principle are completely emptied (one hour for the solution containing 5 mg of particles, two hours for the solution containing 10 mg of particles).

On the contrary, the concentration of SA in the control solution diminishes after every removal.

In particular, considering that there was 1 mg of SA in 0.5 ml of the saturated solution removed, the solution containing 5 mg of mesoporous particles that therefore carry 1 mg of SA (20% of 5 mg), has a stable concentration for the first hour and only after the second removal does it begin to diminish (squares).

The solution containing 10 mg of mesoporous silica that therefore carries 2 mg of SA (20% or 10 mg) has a stable concentration for the first two hours, starting to diminish only after the third removal (circles).

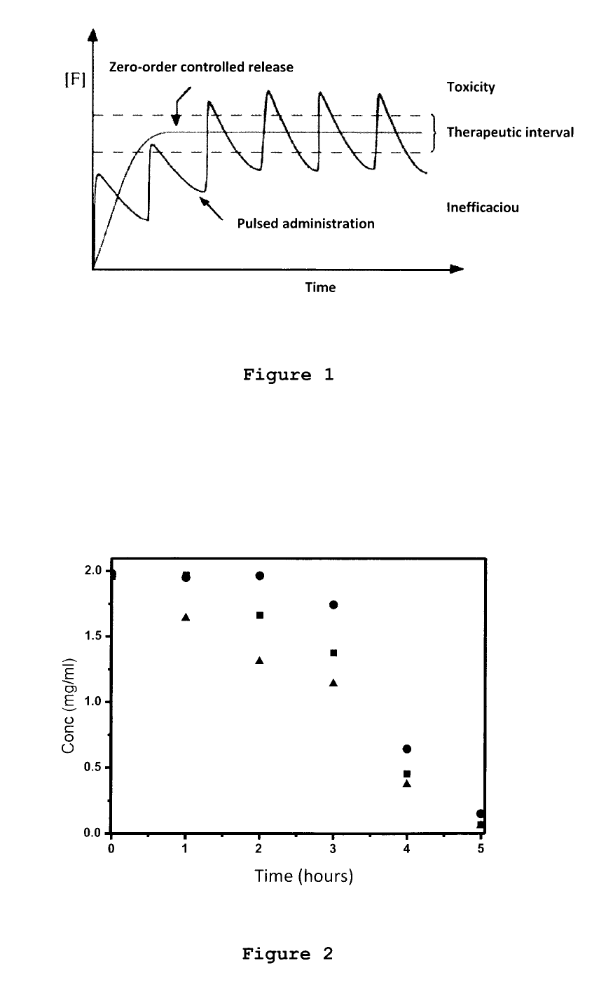

Thus it is clear that by modifying the amount of mesoporous silica particles present in the composition it is possible to program the period of time in which the active principle is released at a constant concentration obtaining the type of zero order release described in FIG. 1.

Naturally, while the principles of the invention remain constant, the structural details and the embodiments may vary, even appreciably, with reference to what has been described and illustrated by way of example only, without departing from the scope of the present invention.

1. Eudermic composition comprising:
at least one eudermically active principle,
at least one vehicle for said active principle, and
porous silica particles,
wherein said active principle is contained in at least one pore of at least a first portion of said silica particles and in said vehicle.

2. Eudermic composition according to claim 1, comprising at least a further vehicle.

3. Eudermic composition according to claim 1, wherein the porous silica particles are silica particles having sub-micrometric sizes, and wherein the pores of said silica particles have a size comprised in the range 2 nm to 50 nm.

4. Eudermic composition according to claim 1, wherein said active principle is contained in said vehicle at a concentration equal to its saturation concentration in said vehicle.

5. Eudermic composition according to claim 1, wherein said active principle contained in said first portion of said porous silica particles is present in an amount comprised in the range 10% to 50% by weight of said portion.

6. Eudermic composition according to claim 2, wherein said active principle is insoluble or poorly soluble in said at least a further vehicle.

7. Eudermic composition according to claim 2, wherein said composition comprises, in addition, at least a further eudermically active principle, said further active principle being contained in at least one pore of a second portion of said silica particles and in said at least a further vehicle, said further active principle being soluble in said at least a further vehicle.

8. Eudermic composition according to claim 7, wherein said further active principle is contained in said at least a further vehicle at a concentration equal to its saturation concentration in said at least a further vehicle.

9. Eudermic composition according to claim 7, wherein said at least a further active principle is insoluble or poorly soluble in said vehicle for said active principle.

10. Eudermic composition according to claim 1, wherein said vehicle and said at least a further vehicle are an hydrophilic vehicle and/or a lipophilic vehicle.

11. Eudermic composition according to claim 1, wherein said active principle and/or at least a further active principle is steadily released over a period of time between 1 and 10 days.

12. Eudermic composition according to claim 1, wherein said eudermic composition is a dermatological composition or a cosmetic composition.

13. Eudermic composition according to claim 1, wherein said eudermic composition is in the form of cream, pomade, ointment, gel, paste.

14. Eudermic composition according to claim 1, wherein said eudermic composition is contained in a transdermal system release.

15. Eudermic composition according to claim 1 for use in the eudermic treatment of the skin.
